(19) United States
(12) Patent Application Publication
(10) Pub. No.: US 2004/0054305 A1
(43) Pub. Date: Mar. 18, 2004

(54) Method and a device for preventing and treating ligament injuries in the lower extremities

(76) Inventor: Torbjorn Berglund, Tierp (SE)

Correspondence Address: BURNS DOANE SWECKER & MATHIS L L P, POST OFFICE BOX 1404, ALEXANDRIA, VA, 22313-1404, US

(21) Appl. No.: 10/415,072

(22) PCT Filed: Oct. 25, 2001
(86) PCT No.: PCT/SE01/02333

(51) Int. Cl. A61B 5/103

(52) U.S. Cl. 600/592

(57) ABSTRACT

A medical footplate (10) is provided, having a first portion for contacting the heel of a foot of a patient, second portion (22) for contacting a front segment of the foot, and an intermediate third portion for contacting an arch of the foot. The third portion has a smaller width that the first and second portions. The foot plate is adapted to provided a sufficiently unstable support for the foot, to stimulate back and forth movement of the bare foot, in a sideways direction when the person tries to maintain his balance. The method is performed thereby that the person tries to stand on the plate supporting himself by that foot only.

### TECHNICAL FIELD

The present invention relates to a device and method for treating or preventing joint and ligament injuries in the lower extremities, such as a sprained ankle or different types of serious knee injuries, by standing on a medical foot plate. The objects of the invention are to provide methods and devices for efficiently preventing or treating injuries of the indicated types.

These objects, as well as further objects and advantages mentioned in this specification, are partly or fully attained by the device as defined in the appended independent device claims, when used as described herein and by the method as defined in the appended independent method claim. Embodiments of the invention are defined in the appended dependent claims. Basically, the invention refers to a medical foot plate, which is sufficiently unstable to stimulate back and forth movement of a bare foot in a sideways direction, when a person tries to stand on one leg only, and mainly supports himself by the foot standing on the plate.

In preferred embodiments the foot plate is sufficiently narrow to provide this stimulation.

The stimulation can also be provided by making the plate sufficiently resilient to stimulate said back and forth movement of the bare foot in a sideways direction.

In preferred embodiments, the plate is made of a rubber like material which is deformed by the weight of the person using it, and the plate has a relatively high friction against the person's naked foot in order to prevent slipping.

The inventive method basically resides in that a person tries to stand on one leg only while trying to support his weight on the foot of that leg, when standing on the medical foot plate, that the person tries to maintain his balance when standing on the plate, whereby the foot will be stimulated to perform a back and forth movement in a sideways direction. This movement pattern is advantageous either for preventing and reducing incidents of ligament injuries, or to decrease the rehabilitation time after injury.

Embodiments of the invention will now be described with reference to the appended drawings.

### BRIEF DESCRIPTION OF THE DRAWINGS

FIG. 1 is a perspective view of the medical foot plate of the present invention;

FIG. 2 is a top view of the medical foot plate shown in FIG. 1;

FIG. 3 is an elevational left side view of the medical foot plate shown in FIG. 1;

FIG. 4 is a bottom view of the medical foot plate shown in FIG. 1;

FIG. 5 is an elevational right side view of the medical foot plate shown in FIG. 1;

FIG. 6 is an elevational front view of the medical foot plate shown in FIG. 1;

FIG. 7 is an elevational rear view of the medical foot plate shown in FIG. 1;

FIG. 8 is a front view of the medical foot plate shown in FIG. 1 with a person standing with his left foot thereon;

FIG. 9 is a front view of the medical foot plate shown in FIG. 1 with a person standing with his right foot thereon:

FIG. 10 is an elevational side view with one foot placed thereon:

FIG. 11 is a front view of the medical foot plate with a foot placed thereon along line 11-11 of FIG. 10; and

FIG. 12 is a front view of the medical foot plate with a foot placed thereon along line 11-11 of FIG. 10.

### DETAILED DESCRIPTION

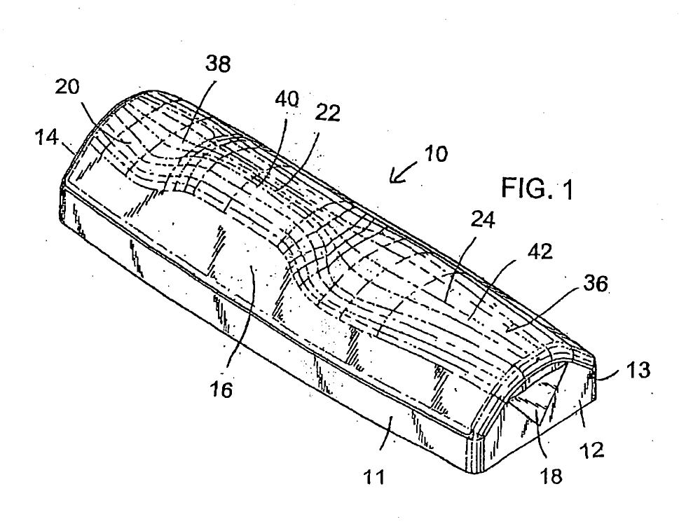

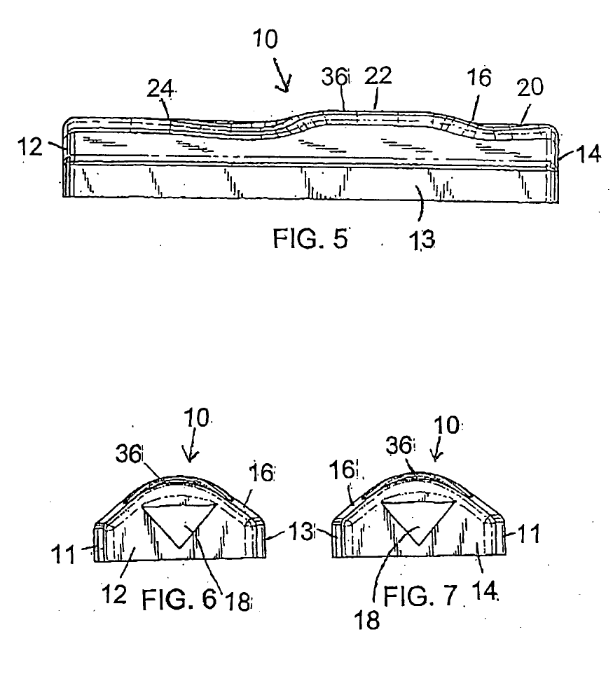

With reference to FIGS. 1-12, the present invention is a medical foot plate or a “balance board” 10 that has a front side 12 and a back side 14 with a soft upper side 16 that is shaped according to a human foot (anatomical shape). The plate 10 is particularly useful for putting stress on the proprioceptive system, i.e. facilitate and stimulate the sense for positions. Lots of studies have observed a decrease in the ability to maintain single leg stance in the sprained ankle as compared to the contralateral uninjured ankle. Injuries to capsuloligamentous structures in the knee joint have resulted in alterations in kinaesthesia the sense for the position of the joint. These findings show that the injuries may not only cause damage to supporting ligamentous joint capsule and muscle structures, but also to different types of neural endings. The loss of neural input from ligament and joint mechanoreceptors may lead to a longer rehabilitation period and a higher risk for reinjuries.

To summarize many studies in this field, it seems reasonable to practice balance training, both for preventing and reducing the incidence of ligament injuries in the lower extremities and to decrease the rehabilitation time after the injury.

The plate 10 has opposite vertical sides 11, 13 that extend between the front side 12 and the back side 14. The front side 12 has a triangular shaped opening 18 defined therein. Of course, the opening may have any suitable shape such as round or oval shaped. The opening 18 extends through the plate 10 so that the back side 14 also has the opening 18 defined therein.

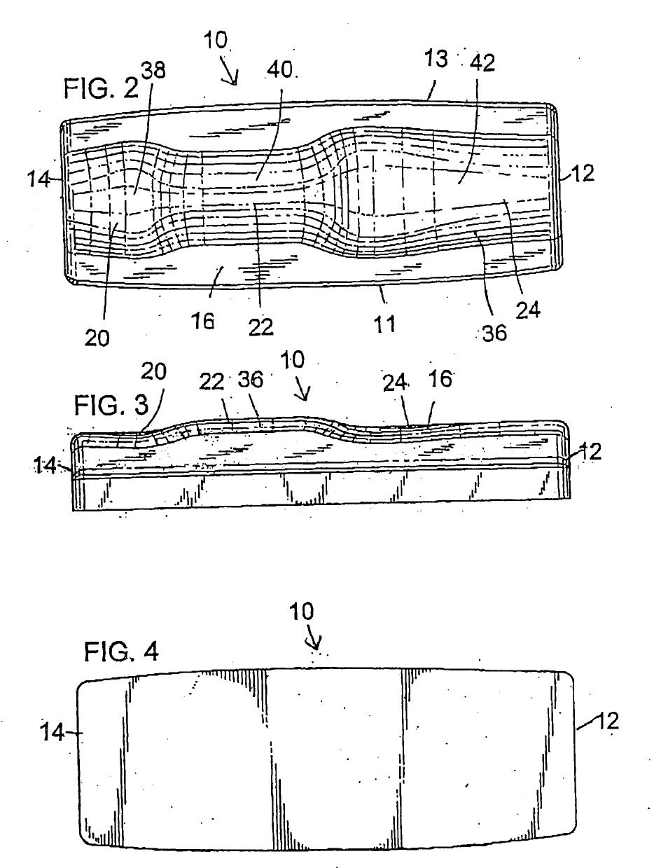

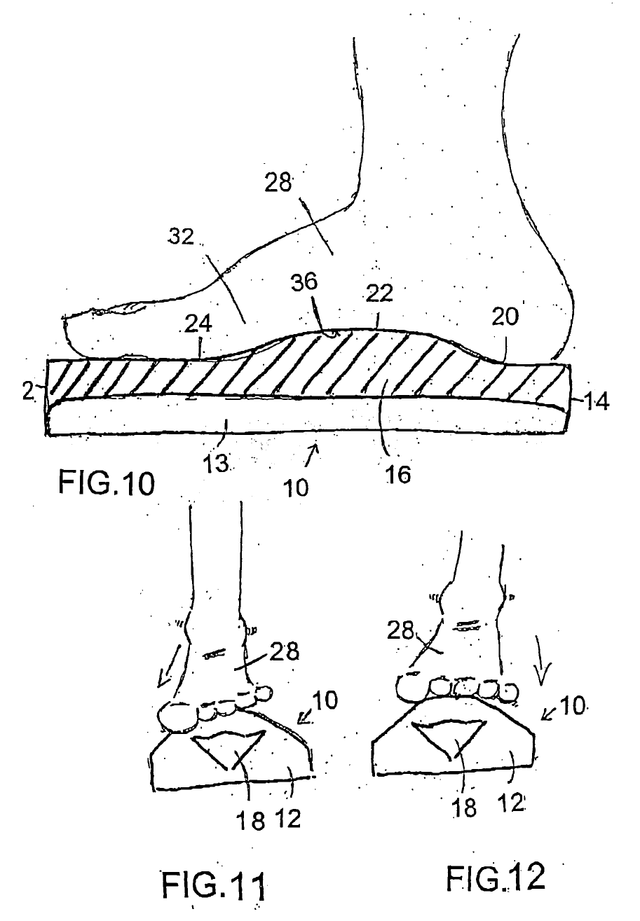

The upper side 16 has a back portion 20, a raised middle portion 22 and a front portion 24. The back portion 20 is formed to receive a heel 26 of a foot 28 (see FIG. 10) and the middle portion 22 supports an arch 30 of the foot 28, while the front portion 24 supports a front segment 32 of the foot 28, s0 that the plate 10 is ergonomically adapted to the shape of the foot 28. A top segment 34 of the upper side 16 has a high friction surface 36, so that the foot 28 does not slip on the plate 10 when a person is standing with one foot balancing on the plate 10. The surface 36 should be sufficiently narrow to stimulate back and forth movement of the foot in a sideways direction when a person stands on the surface 36. The surface 36 has a relatively wide back segment 38 and narrow middle segment 40 and a wide front segment 42, as best shown in FIG. 2.

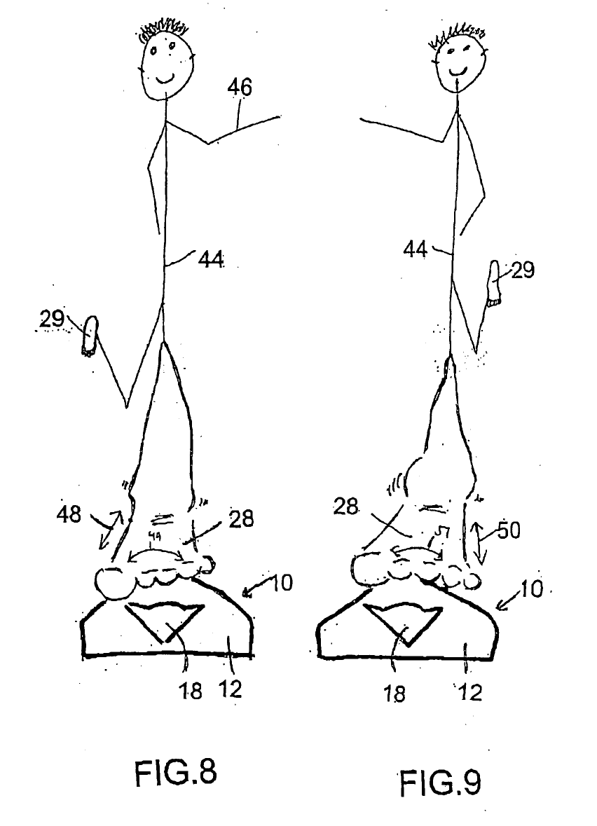

In operation, a person 44 stands bare feet, so that bare foot 28 is placed on the plate 10 and then lifts up the other foot and stretches out one of his arms 46, so that the foot 28 will move back and forth sideways, as shown by double arrows 48, 49, 50 and 51 in FIGS. 8-9, as the person tries to gain the balance while standing on the relatively narrow plate 10. The foot 29 that is not treated may first be very close to the floor, so that the person 44 may gain some balance as the foot 28 is supported by the plate 10. The person 44 may then be lifted up the other foot and the arm 46 is stretched out to shift the point of gravity of the body 44. The high friction area 36 of the plate 10 reduces the risk that the foot 28 is going to slide off the plate. Because the plate 10 is made of a rubber-like material, such as polyurethane, the foot 28 may deform the plate 10 and the plate 10 is not absolutely stable so that the foot will move back and forth in a sideways direction. The opening 18 further weakens the stability of the upper part of the plate 10 to encourage the foot to wiggle back and forth on the plate 10.

In the embodiment according to FIGS. 1-7, the back portion 20 supports the heel 26 over a width of about 4 cm. Also the front portion 24 supports the foot over an effective width of about 4 cm.

The plate 10 supports the foot about 4 cm above the surface on which the plate rests.

In a tested embodiment, the plate consists of a material PU 80, that is a polyurethane having the hardness 80. In practice, this material is compressed about 0.5 cm by a person having a weight of 80 kg when the plate has a thickness of about 40 mm, and a through-going bole 18 as per FIGS. 6-7.

The cross section of the hole 18 is generally a like sided triangle, having a top pointing toward the bottom surface of the plate, and a side parallel to the bottom surface, and located centrally in the plate. The side length of the triangular cross section is about 3 cm. The hole reduces the stability of the plate part above the hole, to stimulate sideways movement of the foot. When the plate has a hole 18, the material of the plate could be chosen not to exhibit any significant elastical compression under the influence of the weight of the user. But if the plate lacks the hole 18, significant elastical compressibility of the plate could improve the stimulation of sideways movement of the foot.

The middle portion 22 has narrow support surfaces against the arch of the foot so as not to prevent sideways rolling motion of the foot while supporting the arch. The medial and lateral surfaces flanking the effective foot supporting areas of the plate slope downwards from the foot support areas.

1. A device for treating or preventing joint or ligament injuries in the lower extremities of a patient, characterized by a medical foot plate having a top surface area, which is adapted to contact the lower side of a foot of a patient, said surface extending in a longitudinal direction of the plate, said plate having a first portion (20) for contacting the heel of the foot, and a second portion (24) for contacting a front segment of the foot, said first, and second portions (20, 24) being arranged sufficiently unstable to stimulate a back and forth movement of the bare foot in a sideways direction when the person tries to support himself on the plate by means of one foot only.

2. A device according to claim 1, characterized in that the first and second portions have a width of about 4 cm.

3. A device according to claims 1 or 2, characterized in that the plate mainly consists of an elastomeric material.

4. A device according to claim 3, characterized in that the elastomeric material is polyurethane PU 80.

5. A device according to any of claims 1-4, characterized in that the plate has a longitudinally extending hole having a width corresponding to the effective width of said first and second portions.

6. A device according to any of claims 1-5, characterized in that the top surface of the plate has a third portion (22) intermediate the first and second portions, for contacting the arch of the foot, the third portion supporting the arch over a width which is smaller than the support width of said first and second portions.

7. A device according to any of claims 1-6, characterized in that the device is mirror symmetrical in relation to a plane which is perpendicular to a support plane of the plate and parallel to the longitudinal direction.

8. A device according to any of claims 1-7, wherein the top of the plate has a high friction surface.

9. A method for preventing or treating a joint or ligament injury in the lower extremities of a person, wherein the person places a bare foot on a plate according to any of claims 1-7 and tries to support his weight on the plate by said foot only, whereby the foot will move back and forth sideways when the person tries to maintain his balance.
